(19) United States
(12) Patent Application Publication
(10) Pub. No.: US 2018/0008757 A1
(43) Pub. Date: Jan. 11, 2018

(54) GELLING PRODUCT FOR FLUID-COLLECTING CONTAINER

(71) Applicant: MEDELA HOLDING AG, Baar (CH)

(72) Inventor: Lukas Bannwart, Rotkreuz (CH)

(73) Assignee: Medela Holding AG, Barr (CH)

(21) Appl. No.: 15/543,875

(22) PCT Filed: Jan. 11, 2016
(86) PCT No.: PCT/EP2016/050351

§ 371 Date: Jul. 14, 2017

(30) Foreign Application Priority Data
Jan. 20, 2015 (EP) 15151779.4

(51) Int. Cl. A61M 1/00; D06N 3/04; B65D 33/16; B65D 75/04; B65D 65/46; B65D 75/30; B65D 77/10

(52) U.S. Cl. CPC A61M 1/0096; B65D 75/30; D06N 3/04; B65D 77/10; A61M 1/0001; B65D 75/04; B65D 65/46; B65D 33/1641; D06N 2209/1621

(57) ABSTRACT

A gelling product for solidifying a body fluid aspirated in a fluid collection container has an interior, a gelling agent arranged in the interior, and a water-soluble cover. The water-soluble cover is made of a textile material. The gelling product, in particular a gelling bag, is in this way prevented from bursting too early.

### CROSS-REFERENCE TO RELATED APPLICATIONS

The present application is the US national phase of International Application No. PCT/EP2016/050351, filed Jan. 11, 2016, which application claims priority to European Application No. 15151779.4 filed Jan. 20, 2015. The priority application, EP 15151779.4, is hereby incorporated by reference.

### TECHNICAL FIELD

The present invention relates to a gelling product for solidifying a body fluid that has been aspirated in a fluid collection container.

### PRIOR ART

Stationary or mobile suction systems are used in the medical field to aspirate body fluids or secretions from body cavities or wounds, particularly in thorax drainage. These systems in each case comprise a suction source, for example a vacuum pump or a central vacuum, and one or more drainage containers. A vacuum line connects the drainage container to the suction source, and a drainage line leads from the drainage container to the patient. The applied vacuum generates an underpressure in the container, and the fluid that is to be aspirated, or the secretion that is to be aspirated, is aspirated from the patient into the drainage container and collected there. The drainage container can be stiff or it can be a soft, flexible bag. If a bag is used, the latter can be arranged in turn in a stiff outer container that can preferably be closed in an airtight manner.

A container filled with aspirated body fluid has to be disposed of in the correct way. This is especially the case if blood is aspirated. During transport and disposal of the container, however, in order to ensure that no fluid can escape from the container and in order to minimize the risk of infection, the fluid in the container is already solidified or thickened to a viscous gel. Containers with gelled fluid are covered by a different disposal class than containers with non-solidified contents. Disposal is simpler and cheaper.

A granular gelling material of this kind is known, for example, from U.S. Pat. No. 5,595,731. In order to ensure that the powder or granules cannot get into narrow areas of the container or even into the drainage tubes prior to use, it is usually also packed.

U.S. Pat. No. 5,307,819, U.S. Pat. No. 5,234,419, U.S. Pat. No. 5,185,007, U.S. Pat. No. 5,279,602 and WO 2010/006458 disclose partition walls between an area containing a gelling agent and the rest of the interior of a fluid collection container, wherein the partition walls are actively removed by the user. However, if the hospital staff forget to remove the partition wall, the fluid remains unthickened.

U.S. Pat. No. 6,615,862 discloses a floating ball with a water-soluble foil bag arranged therein.

U.S. Pat. No. 5,238,582 discloses a rigid drainage container in which a bag with a gelling agent is suspended. The outer sleeve of the bag is either a water-soluble foil or a water-permeable woven fabric or a water-permeable fibrous material.

WO 2009/144486 and WO 2013/030581 disclose a drainage bag in which a small bag or a sachet filled with gelling granules is loosely placed. The small bag or the sachet has a sleeve made of water-soluble paper. In other cases, these gelling bags have an outer sleeve made of a water-soluble foil.

Gelling products with water-soluble outer sleeves are inexpensive and can be widely used. However, they have the disadvantage that they often burst even before use. Hospitals that find burst gelling bags in individual fluid collection containers tend to send the entire batch back to the manufacturer.

### DISCLOSURE OF THE INVENTION

It is therefore an object of the invention to make available an inexpensive gelling product in which the danger of the outer sleeve bursting is minimized.

The gelling product according to the invention, for solidifying a body fluid aspirated in a fluid collection container, has an interior, a gelling agent arranged in the interior, and a water-soluble cover. According to the invention, the water-soluble cover is made of a textile material.

It was found that bags burst more easily if they have been exposed previously to an elevated temperature and an elevated atmospheric humidity. This is the case, for example, when the fluid collection container with the gelling product placed in it is sterilized, e.g. sterilized with ethylene oxide (ETO). In this type of sterilization, the gelling product is typically exposed to temperatures of 50° C. and to an atmospheric humidity of 70%. The gelling or solidifying agent begins to swell. If the outer sleeve is a water-soluble foil, the latter shrinks as a result of the temperature or of the temperature difference. This can result in a hard bag. At colder temperatures, particularly at below-zero temperatures in the winter or in premises that are excessively cooled, the foil becomes brittle and may even burst under a slight impact.

This can be prevented by virtue of the use, according to the invention, of a water-soluble textile material for the cover, since the material does not become brittle. If the material has sufficient elasticity, this additionally prevents the bag from becoming hard when the gelling agent has swollen slightly.

Before use, the gelling product according to the invention can therefore be easily sterilized together with the fluid collection container.

The term “textile material” is used here in the usual sense. That is to say, it designates a two-dimensional structure that was constructed from fibres and/or threads. The textile material can be a nonwoven or felt, for example. However, it can also be a woven or knitted fabric, for example. It can be made from a natural starting material, a plastic, or a mixture thereof.

The feature “water-soluble” signifies that the material dissolves on contact with water or a water-containing fluid in the temperature range present during the use of the gelling product. The cover according to the invention preferably dissolves in cold water or at least on contact with water at the room temperature range. The cover according to the invention is preferably optimized in terms of the choice of material, such that it dissolves within the predetermined time interval during the planned use.

In a preferred embodiment, the water-soluble cover is made of PVA (polyvinyl acetate). Preferably, the water-soluble cover according to the invention is made of a nonwoven, in particular a PVA nonwoven. However, the other abovementioned embodiments of textile materials can also be used.

In one embodiment, the gelling product is composed only of the gelling agent and of said textile cover. In another embodiment, a second cover is present which is made of a water-soluble and water-impermeable material and which is arranged between the water-soluble textile cover and the gelling agent. This second cover is preferably composed of a foil, in particular of PVAL (polyvinyl alcohol). The second cover can be rigidly connected to the first cover, for example forming a two-layer composite material, or it can be configured as a self-contained and independent element.

The gelling agent is usually a known type of granular material, for example a swellable, water-insoluble crosslinked polymer. However, other types of thickening agents or solidifying agents can also be used.

In a preferred illustrative embodiment, the water-soluble cover completely surrounds the gelling agent. The cover preferably forms a bag in which the gelling agent is held. This bag can be arranged preferably loosely in a fluid collection container or can be secured in the latter. The bag can be used in stiff fluid collection containers and also in soft, flexible fluid collection bags.

If the gelling product is designed as a bag, it preferably has at least one closure zone for forming a closed cavity, which forms the interior for receiving the gelling agent. The bag can be produced from a multi-part textile cover. However, the cover is preferably in one part or at most two parts.

The closure zone can have different configurations depending on the choice of material and on the shape of the gelling product. The closure zone is preferably a seal which firmly connects two layers of the material of the cover or firmly connects one layer of the cover to another material. The connection has to ensure that gelling agent in the form of powder or granules does not escape too early from the interior.

Alternatively or additionally, however, the closure zone can be a seam, an adhesively bonded zone or a weld seam.

The gelling bag can have any desired shape. Flat rectangular or oval shapes, with two transverse closure zones at the ends, have proven useful. If the gelling bag is made from a single piece of material or nonwoven fabric, it usually has a single closure zone in its longitudinal direction. The closure zones at the ends can be rectilinear or curved.

In other embodiments, the bag has a round or rectangular cross section, wherein a closure zone is present which extends around the entire circumference of the bag and is in itself closed. This configuration can be formed, for example, by a process in which two approximately identically shaped materials or nonwovens are placed one over the other and are sealed onto each other.

In another embodiment, the bag is formed from a planar material folded only once, wherein a closure zone is present which has two free ends. The side edge of the bag lying between the ends is already closed by the fold itself.

In a further embodiment, the bag has a torus shape. In another embodiment, the bag is shaped like a pouch and is closed with a clip or staple.

For transport and storage, the gelling product according to the invention is packed in a moisture-proof manner preferably individually, or together with identical gelling products, for example in foil pouches or in lined cardboard boxes.

The gelling product according to the invention can be designed as a body in which only part of its outer sleeve is formed by the water-soluble cover and the remaining area is water-insoluble. Its interior can be formed, for example, by a rigid hollow cylinder, a rear wall, and a front wall spaced apart from the rear wall, wherein the front wall is formed by a water-soluble cover.

A gelling product of this kind, with a water-soluble outer sleeve only in part, can be placed as an autonomous element into a container or can be secured therein. However, it can also be a fixed and in particular integral constituent part of a fluid collection container of a drainage device for aspirating a body fluid.

The invention therefore further comprises a fluid collection container for receiving an aspirated body fluid, wherein the fluid collection container has a receiving space for receiving the aspirated body fluid and a gelling product of this kind, wherein the water-soluble cover forms a partition wall between the interior of the gelling product and the receiving space, and wherein the rigid hollow cylinder and/or the rear wall is configured in one piece with a wall of the fluid collection container surrounding the gelling product.

The invention further comprises a fluid collection container for receiving an aspirated body fluid, wherein the fluid collection container has a receiving space for receiving the aspirated body fluid and an above-described gelling product, wherein the gelling product is arranged in what is a lower area of the receiving space in the position of use of the fluid collection container. The gelling product can be a loosely inserted bag or a loosely inserted capsule with an outer sleeve that is stiff except for the water-soluble cover. The gelling product can also be secured on the fluid collection container, and it can in particular form part of the wall of the fluid collection container.

The gelling product according to the invention prevents early bursting of the outer sleeve.

### BRIEF DESCRIPTION OF THE DRAWINGS

Preferred embodiments of the invention are described below with reference to the drawings, which serve only for illustration and are not to be interpreted as limiting the invention. In the drawings:

FIG. 1 shows a stiff drainage fluid collection container with an inserted gelling bag according to the invention;

FIG. 2 shows a drainage fluid collection bag with flexible bag and rigid lid, and with an inserted gelling bag according to the invention;

FIG. 3 shows a perspective view of a gelling bag according to the invention in a first embodiment;

FIG. 4 shows a plan view of the gelling bag according to FIG. 3;

FIG. 5 shows a partial section through a gelling bag in a second embodiment according to the invention;

FIG. 6 shows a plan view of the gelling bag according to FIG. 5;

FIG. 7 shows a plan view of a gelling bag according to the invention in a third embodiment;

FIG. 8 shows a partially sectioned perspective view of a gelling bag according to the invention in a fourth embodiment;

FIG. 9 shows a partially sectioned perspective view of a gelling bag according to the invention in a fifth embodiment;

FIG. 10 shows a partially sectioned perspective view of a gelling bag according to the invention in a sixth embodiment;

FIG. 11 shows a partially sectioned perspective view of a gelling bag according to the invention in a seventh embodiment;

FIG. 12 shows a partially sectioned perspective view of a gelling bag according to the invention in an eighth embodiment;

FIG. 13 shows a partially sectioned perspective view of a gelling bag according to the invention in a ninth embodiment;

FIG. 14 shows a perspective view of a gelling bag according to the invention in a tenth embodiment;

FIG. 15 shows a partially sectioned perspective view of the gelling bag according to FIG. 14;

FIG. 16 shows a partially sectioned perspective view of a gelling bag according to the invention in an eleventh embodiment;

FIG. 17 shows a longitudinal section through a gelling bag according to the invention in a twelfth embodiment;

FIG. 18 shows a perspective view of part of a stiff fluid collection container with a gelling product according to the invention in a twelfth embodiment;

FIG. 19 shows a longitudinal section through the container part according to FIG. 18;

FIG. 20 shows a partial section through the container part according to FIG. 18, and

FIG. 21 shows a partial section through the gelling product and an adjacent area of the container part according to FIG. 18.

### DESCRIPTION OF PREFERRED EMBODIMENTS

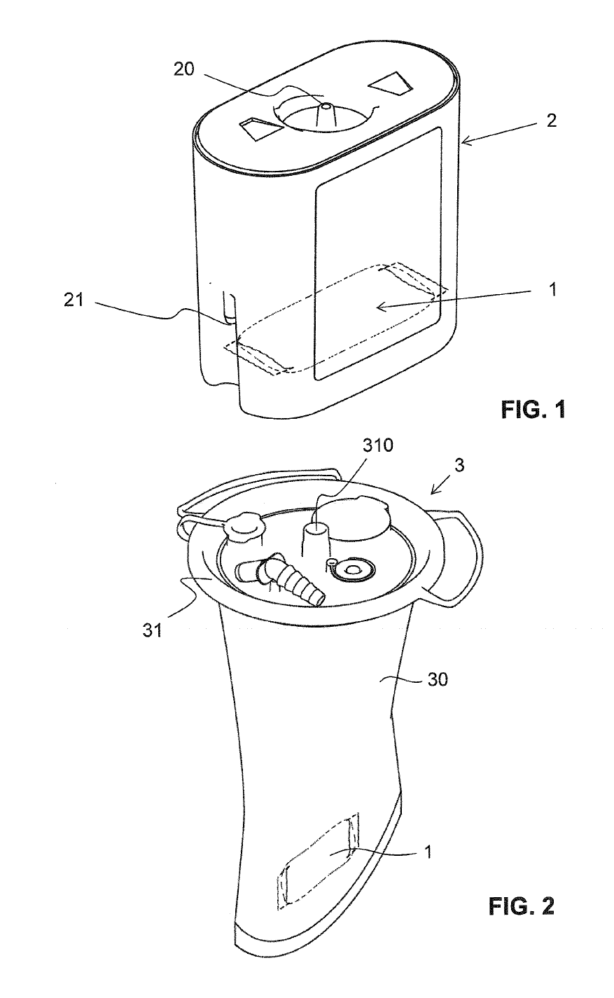

FIG. 1 shows a known drainage fluid collection container 2, e.g. of the kind used together with a thorax drainage pump. It is designed to be used once only and is usually disposed of together with the body fluids pumped from the patient.

The container 2 is preferably produced from a stiff or rigid plastic. It has a drainage port 20 for connection to a suction line leading to the patient, and a suction port 21 for connection to a vacuum source, for example the drainage pump. Otherwise, the container 2 is preferably tightly closed.

A gelling product according to the invention, here in the form of a gelling bag 1, is placed in the hollow receiving space of the container 2. It is preferably placed loosely, i.e. not secured to the container 2.

FIG. 2 shows a drainage bag arrangement 3 of a known type. It has a flexible drainage bag 30 made of plastic, and a closure lid 31. The drainage bag 30 is connected rigidly to the closure lid 31 and cannot be detached without being destroyed. The closure lid 31 has a drainage port 310 for connection to a patient-side drainage line, and a suction port (not visible here in the figure) for connection to a suction source. During use, this drainage bag arrangement is usually held in a rigid outer container (not shown here), with the closure lid 31 resting on the upper edge of the outer container. After it has been filled for the first time with body fluid, this drainage bag arrangement 3 is also disposed of together with said body fluid. As can be seen from FIG. 2, it too has a loosely placed gelling bag 1 not secured to the drainage bag or to the lid.

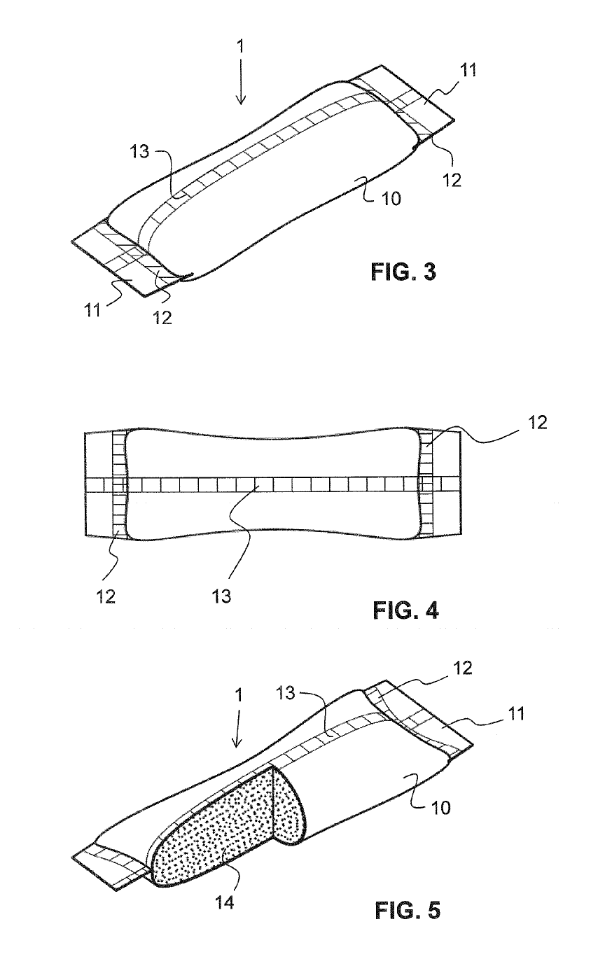

FIGS. 3 and 4 show a first illustrative embodiment of a gelling bag 1 of the kind that can be used in the containers according to FIGS. 1 and 2. It has a sleeve or cover made of a one-part or multi-part water-soluble textile material. The material has closure zones, here sealed areas, such that a closed interior is formed for receiving a known gelling agent, particularly in the form of granules. In the embodiment shown here, a one-part nonwoven, for example a PVA nonwoven, is folded twice in the longitudinal direction and sealed in the longitudinal direction. The corresponding seam is designated by reference number 13. At each of the two ends there is a transversely extending seal 12, with an unsealed edge protruding at both ends. A main body 10 with said interior is thus obtained between the two seals at the ends. As can be seen clearly in FIG. 4, the seals 12 at the ends extend in a straight line. This gelling bag 1 can be produced automatically and relatively easily from a web of material, or from a tube of material, and filled. As was mentioned at the outset, other types of connections can also be used instead of seals.

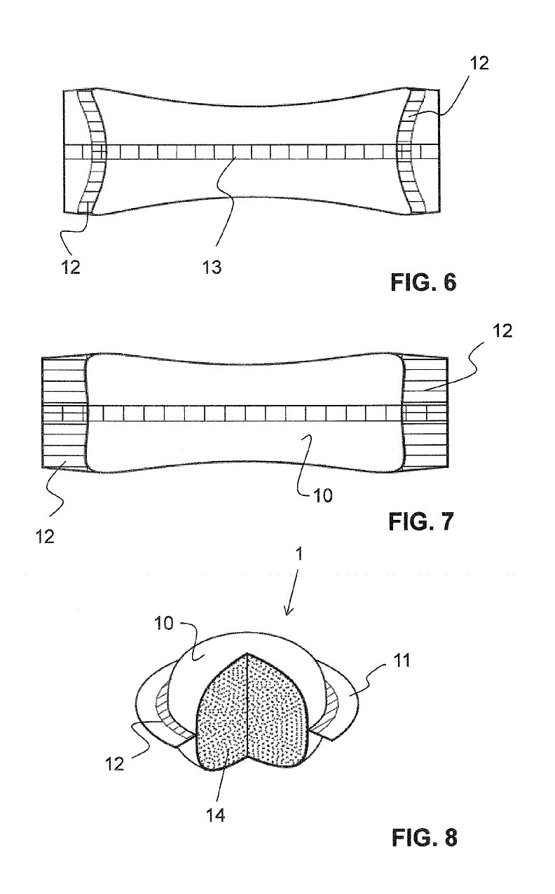

The embodiment according to FIGS. 5 and 6 differs from the previous one only in terms of the shape of the seals 12 at the ends. The seals 12 are curved here. FIG. 5 additionally shows the gelling agent, designated by reference sign 14.

In the embodiment according to FIG. 7, the seals 12 at the ends extend over the entire edge as far as the respective free end.

The gelling bag 1 according to FIG. 8 has a round cross section and is designed like a sphere with flattened poles. A peripheral and closed seal 12 is present here.

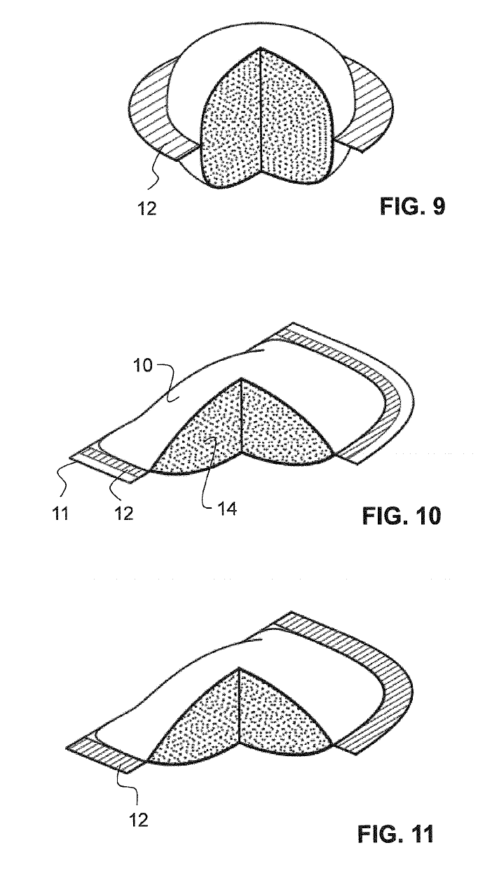

The gelling bag 1 according to FIG. 9 corresponds to that of FIG. 8, with the seal 12 here extending once again as far as the free end of the edge 11.

The embodiments according to FIGS. 10 and 11 are formed by a process in which a one-piece cover has been folded just once, such that an edge of the gelling bag 1 is formed by the material itself. The sealing seam 12 is therefore not in itself closed and instead has two free ends at this edge. The difference between FIGS. 10 and 11 lies once again in the width of the seal 12.

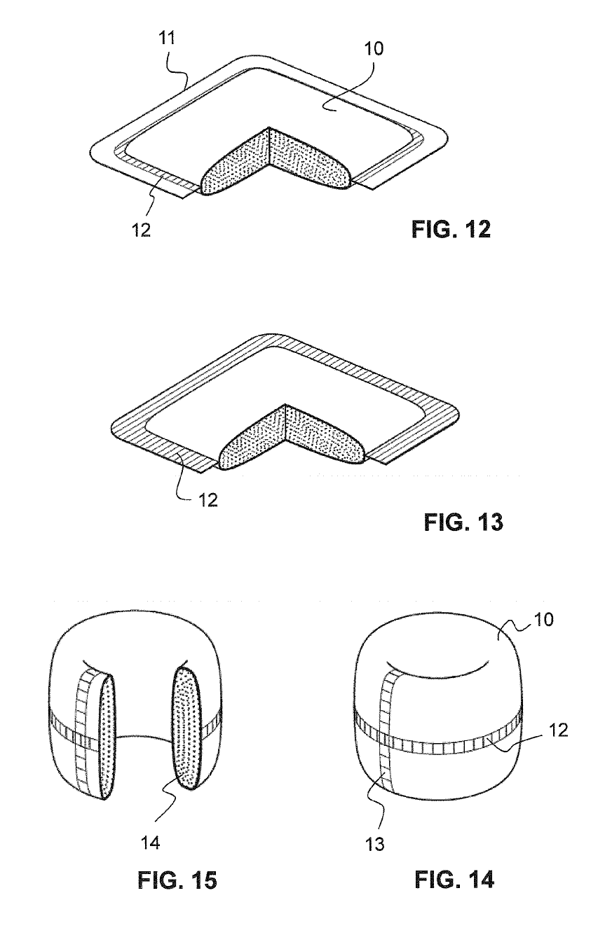

In FIGS. 12 and 13, the shape of the cross section of the gelling bag 1 and of the main body 10 is once again rectangular with rounded corners. Here, however, the seal 12 extends about the circumference and is in itself closed. It can once again terminate at a distance from the free ends of the edges 11 or extend over the entire width of the latter. In these examples, the cover is preferably in two parts, wherein the two parts are placed one over the other and connected to each other. In particular, they can be welded, sewn, sealed or adhesively bonded to each other.

In the embodiment according to FIGS. 14 and 15, the gelling bag 1 has a torus shape.

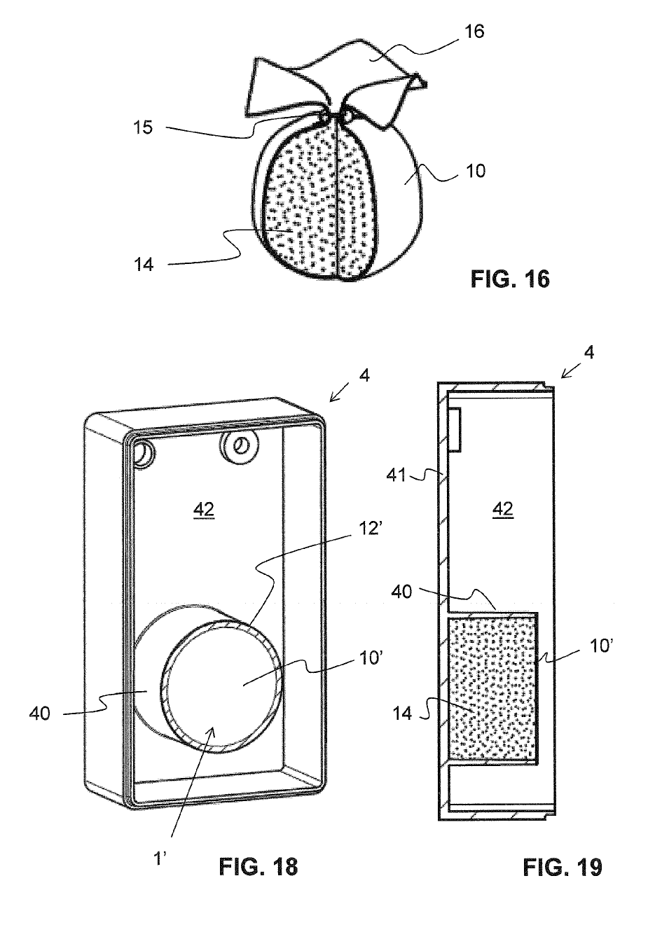

FIG. 16 shows a gelling bag 1 in which the cover is gathered together and held together with a clip or another securing means. The remaining part of the cover that does not form the main body 10 is provided with reference sign 16 in the figure. A seal or another connection may be present but does not have to be.

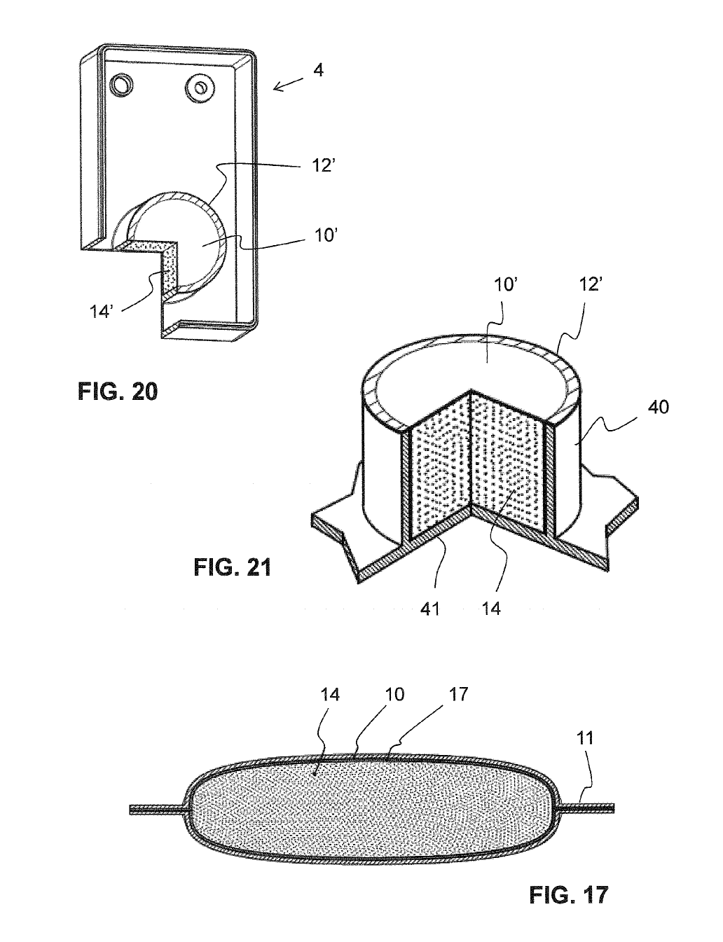

FIG. 17 shows a further variant of a gelling bag according to the invention. The bag is formed by a two-ply material, which completely surrounds the gelling agent 14. On the outside, it is formed by the above-described water-soluble textile first cover 10. On the inside, there is a second cover, which is water-soluble but water-impermeable. The second cover 17 is preferably a foil. The first cover 10 is again preferably made from a nonwoven, preferably from PVA. The water-soluble but water-impermeable second cover 17 is preferably produced from a PVAL foil. Both covers 10, 17 together form the edge 11. They can be arranged loosely one over the other, i.e. can both be formed as bags. However, they can also form a common composite material and can be shaped to give a single bag.

A further gelling product 1′ is shown in FIGS. 18 to 21. Here, the cover does not fully encapsulate the gelling agent 14. The gelling product 1′ has a stiff or rigid body, which is closed on one side by the water-soluble cover 10′. The body is formed in this example by a hollow cylinder 40 and a rear wall 41. The end of the hollow cylinder 40 facing away from the rear wall 41 is covered by the cover 10′. The rear wall 41 is part of a stiff fluid collection container 4 of a drainage device. The gelling product 1′ is preferably arranged in the lower area of the fluid collection container 4.

In other respects, the fluid collection container 4 corresponds, for example, to the container as disclosed in WO 2010/006458, mentioned in the introduction. Only one half of the container is shown in FIGS. 18 to 20. Thus, the receiving space 42 for the aspirated body fluid is not shown closed. The mating piece for closing the receiving space 42 and the container is not shown but corresponds to the mating piece according to WO 2010/006458.

As can be seen from FIG. 19, the hollow cylinder 40 is preferably formed in one piece with the rear wall 41 of the container 4. They are preferably produced from plastic. However, the hollow cylinder 40 and the part of the rear wall 41 closing the cylinder 40 can also be configured as a one-part or multi-part insert piece and can be inserted in a fluid-tight manner into the rest of the rear wall. Moreover, instead of a cylinder shape, it is also possible to choose another shape, for example a cuboid or rectangular shape.

The closure zone, here a seal, of the cover 10′ on the cylinder 40 is designated by reference sign 12′.

As can be seen clearly from FIGS. 20 and 21, the gelling agent 14, particularly in the form of granules, is again arranged in the interior of the gelling product 1′. In this example too, when the canister is filled with body fluids, the cover 10′ is in contact with the fluid and accordingly dissolves, such that the gelling agent 14 is released and the fluid is thickened.

What is claimed is:

1. A gelling product for solidifying a body fluid aspirated in a fluid collection container wherein the gelling product has an interior, a gelling agent arranged in the interior, and a water-soluble cover, wherein the water-soluble cover is made of a textile material.

2. The gelling product according to claim 1, wherein the water-soluble cover is made of a nonwoven.

3. The gelling product according to claim 1, wherein the water-soluble cover is made of PVA (polyvinyl acetate).

4. The gelling product according to claim 1, wherein a second cover is present, which is made of a water-soluble and water-impermeable material and which is arranged between the water-soluble textile cover and the gelling agent.

5. The gelling product according to claim 1, wherein the water-soluble textile cover completely surrounds the gelling agent.

6. The gelling product according to claim 1, wherein the water-soluble textile cover is a bag.

7. The gelling product according to claim 6, wherein the bag has a substantially rectangular shape with two closure zones arranged at the ends in order to form a closed cavity, which forms the interior.

8. The gelling product according to claim 6, wherein the bag has a round or rectangular cross section, wherein a closure zone is present which extends around the entire circumference of the bag and is in itself closed, wherein the closure zone forms a closed cavity, which forms the interior.

9. The gelling product according to claim 6, wherein the bag is formed from a planar material folded only once, and wherein a closure zone is present which has two free ends, wherein the closure zone forms a closed cavity, which forms the interior.

10. The gelling product according to claim 6, wherein the bag has a torus shape.

11. The gelling product according to claim 6, wherein the bag is pouch-shaped and closed with one of a clip or a staple.

12. The gelling product according to claim 1, wherein the interior is formed by a rigid hollow cylinder, a rear wall, and a front wall spaced apart from the rear wall, wherein the front wall is formed by the water-soluble cover.

13. The gelling product according to claim 12, wherein the gelling product is a constituent part of a fluid collection container of a drainage device for aspirating a body fluid.

14. A fluid collection container for receiving an aspirated body fluid, wherein the fluid collection container has a receiving space for receiving the aspirated body fluid and the gelling product according to claim 13, wherein the water-soluble cover forms a partition wall between the interior of the gelling product and the receiving space, and wherein at least one of the rigid hollow cylinder and the rear wall is configured in one piece with a wall of the fluid collection container surrounding the gelling product.

15. A fluid collection container for receiving an aspirated body fluid, wherein the fluid collection container has a receiving space for receiving the aspirated body fluid and the gelling product according to claim 1, wherein the gelling product is arranged in what is a lower area of the receiving space in the position of use of the fluid collection container.
